(19) United States
(12) Patent Application Publication
(10) Pub. No.: US 2025/0235098 A1
(43) Pub. Date: Jul. 24, 2025

(54) EYE USAGE PATTERN DETERMINATION AND EVALUATION

(71) Applicant: Dell Products L.P., Round Rock, TX (US)

(72) Inventors: Venkata Nagendra Purushotham Musti, Hyderabad (IN); Girish VenkateshaMurthy, Bengaluru (IN); Sai Shashank Akula, Khammam (IN)

(21) Appl. No.: 18/420,944

(22) Filed: Jan. 24, 2024

(51) Int. Cl. A61B 3/113; A61B 90/00; G06T 7/20

(52) U.S. Cl. CPC A61B 3/113; A61B 90/361; G06T 7/20; G06T 2207/10016; G06T 2207/30041

(57) ABSTRACT

An apparatus comprises at least one processing device configured to obtain eye tracking data associated with a computing device and to identify, based at least in part on the obtained eye tracking data, presence of a user viewing a display screen associated with the computing device. The at least one processing device is also configured to determine, based at least in part on the obtained eye tracking data, eye usage data of the identified user. The at least one processing device is further configured to process the determined eye usage data to generate at least one data structure characterizing conformance of the identified user with a set of eye usage patterns, to generate one or more recommendations for altering an eye usage of the identified user based at least in part on the generated at least one data structure, and to provide the generated recommendations to the computing device.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

The use of computing devices and other digital tools continues to increase, and such digital tools have proliferated into all walks of life, including personal and professional. Digital tools include, for example, personal computing devices like laptops and desktops, as well as devices such as smartphones, tablets, smartwatches, gaming systems, etc. While such digital tools provide various benefits and positive changes, the use of such digital tools can also present various health-related hazards. Health-related hazards may be a result of, for example, poor sitting posture, poor viewing angles, poor lighting conditions, etc. Prolonged and improper screen viewing habits can contribute to a range of issues and conditions for the eyes, head, neck, shoulders, etc. Ensuring proper eye usage, also referred to as exercising proper eye ergonomics, provides various benefits including mitigating or reducing the risk of developing such issues and conditions.

### SUMMARY

Illustrative embodiments of the present disclosure provide techniques for determining eye usage of a user and for evaluating conformance of the user with a set of eye usage patterns for viewing display screens associated with computing devices.

In one embodiment, an apparatus comprises at least one processing device comprising a processor coupled to a memory. The at least one processing device is configured to obtain eye tracking data associated with a computing device and to identify, based at least in part on the obtained eye tracking data, presence of a user viewing a display screen associated with the computing device. The at least one processing device is also configured to determine, based at least in part on the obtained eye tracking data, eye usage data of the identified user. The at least one processing device is further configured to process the determined eye usage data to generate at least one data structure characterizing conformance of the identified user with a set of one or more eye usage patterns for viewing the display screen associated with the computing device, to generate one or more recommendations for altering an eye usage of the identified user based at least in part on the generated at least one data structure, and to provide the generated one or more recommendations to the computing device.

These and other illustrative embodiments include, without limitation, methods, apparatus, networks, systems and processor-readable storage media.

### BRIEF DESCRIPTION OF THE DRAWINGS

FIG. 1A is a block diagram of an information processing system configured for evaluating eye ergonomics of a user viewing a display screen of a computing device in an illustrative embodiment.

FIG. 1B is another block diagram of an information processing system configured for evaluating eye ergonomics of users viewing display screens of computing devices in an illustrative embodiment.

FIG. 2 is a flow diagram of an exemplary process for determining eye usage of a user and for evaluating conformance of the user with a set of eye usage patterns for viewing display screens in an illustrative embodiment.

FIG. 3 shows a system including a smart eye ergonomics evaluation framework configured to implement checker tools for various aspects of eye ergonomics in an illustrative embodiment.

FIG. 4 shows a laptop computing device configured for use with a smart eye ergonomics evaluation framework monitoring eye distance to a display screen of the laptop computing device in an illustrative embodiment.

FIG. 5 shows a process flow for face recognition in an illustrative embodiment.

FIG. 6 shows visualizations of different eye distances to display screen operating scenarios in an illustrative embodiment.

FIG. 7 shows pseudocode for implementing a screen distance checker tool of a smart eye ergonomics evaluation framework in an illustrative embodiment.

FIG. 8 shows a visualization of different viewing angle ranges to a display screen of a computing device in an illustrative embodiment.

FIGS. 9A and 9B show pseudocode for implementing a viewing angle checker tool of a smart eye ergonomics evaluation framework in an illustrative embodiment.

FIG. 10 shows a visualization of screen time checking in an illustrative embodiment.

FIGS. 11A and 11B show pseudocode for implementing a blink rate checker tool of a smart eye ergonomics evaluation framework in an illustrative embodiment.

FIGS. 12 and 13 show examples of processing platforms that may be utilized to implement at least a portion of an information processing system in illustrative embodiments.

### DETAILED DESCRIPTION

Illustrative embodiments will be described herein with reference to exemplary information processing systems and associated computers, servers, storage devices and other processing devices. It is to be appreciated, however, that embodiments are not restricted to use with the particular illustrative system and device configurations shown. Accordingly, the term “information processing system” as used herein is intended to be broadly construed, so as to encompass, for example, processing systems comprising cloud computing and storage systems, as well as other types of processing systems comprising various combinations of physical and virtual processing resources. An information processing system may therefore comprise, for example, at least one data center or other type of cloud-based system that includes one or more clouds hosting tenants that access cloud resources.

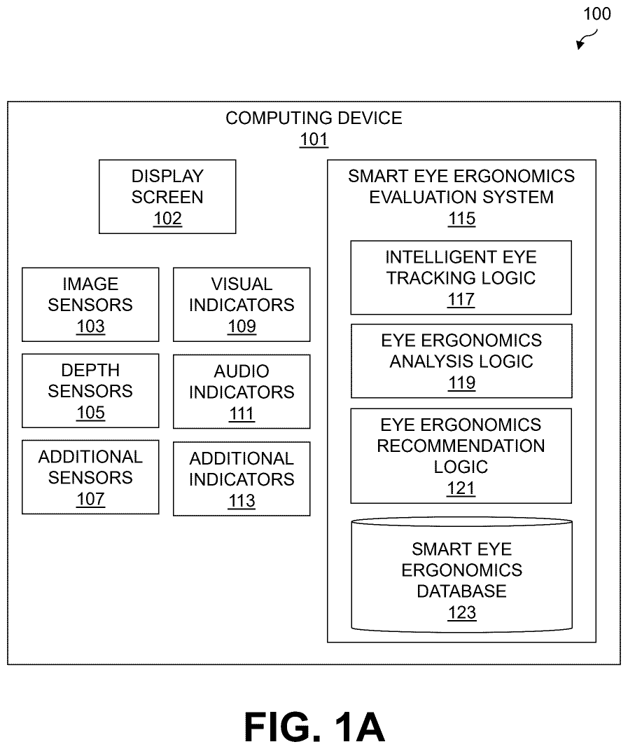

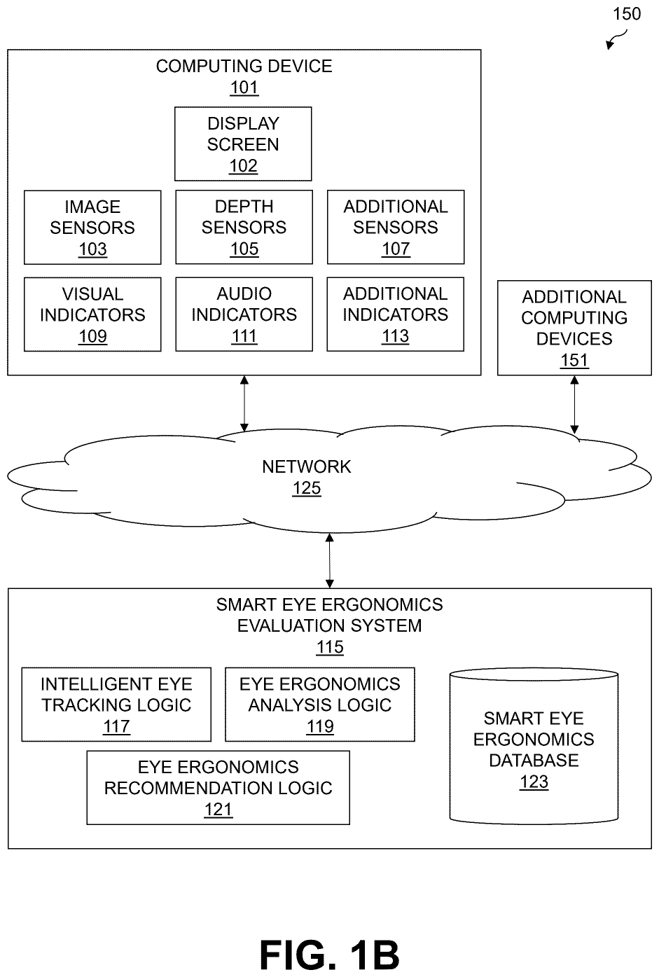

FIGS. 1A and 1B show information processing systems 100 and 150 configured in accordance with illustrative embodiments. The information processing systems 100 and 150 are assumed to be built on at least one processing platform and provides functionality for evaluating eye usage patterns (also referred to as eye ergonomics) of a user viewing a display screen 102 of a computing device 101 and possibly additional computing devices 151.

The computing device 101 may comprise, for example, a laptop, tablet, smartphone, smartwatch, smart glasses etc. (e.g., where the display screen 102 is integrated in the housing thereof), a desktop (e.g., where the display screen 102 is a monitor connected to the desktop), a gaming system (e.g., where the display screen 102 may be integrated therewith or externally connected), etc. The computing device 101 includes or is connected to various sensors, including one or more image sensors 103 (e.g., cameras including integrated or external webcams, front-facing cameras, infrared sensors, etc.), depth sensors 105 and possible additional sensors 107. The computing device 101 also includes or is connected to various indicators, including one or more visual indicators 109 (e.g., one or more light-emitting diodes (LEDs), one or more designated portions of the display screen 102 or an additional display screen separate from the main display screen 102 of the computing device 101, etc.), audio indicators 111 (e.g., speakers, etc.) and additional indicators 113 (e.g., haptic feedback devices, vibration elements, etc.). The additional computing devices 151 shown in FIG. 1B are each assumed to be configured in a manner similar to that of the computing device 101, though it should be appreciated that different ones of the computing device 101 and the additional computing devices 151 may be configured with different sets of sensors and/or indicators.

In the information processing system 100 of FIG. 1A, a smart eye ergonomics (SEE) evaluation system 115 is implemented internal to the computing device 101 while in the information processing system 150 of FIG. 1B, the SEE evaluation system 115 is implemented external to the computing device 101 and the additional computing devices 151. In the information processing system 150 of FIG. 1B, the computing device 101 and the additional computing devices 151 are connected to the SEE evaluation system 115 via one or more networks 125. The embodiment of FIG. 1A is one in which the functionality of the SEE evaluation system 115 is executed “locally” within the computing device 101, which may provide various benefits related to security and privacy concerns as no information (e.g., a video stream used for face recognition and evaluating different aspects of eye usage) needs to be provided outside the computing device 101. Such local processing, however, comes at a cost of increased use of computing resources of the computing device 101, though this may be mitigated through selection of lightweight models for implementing various logic of the SEE evaluation system 115 as described elsewhere herein. The embodiment of FIG. 1B is one in which the functionality of the SEE evaluation system 115 is executed “remote” from the computing device 101 and the additional computing devices 151, which may provide benefits as potentially greater computing resources being available on an information technology (IT) infrastructure (potentially a cloud-based set of servers, or a virtualization infrastructure which may scale on demand) on which the SEE evaluation system 115 runs. Such remote processing, however, also comes at a cost of increased use of network bandwidth and latency (e.g., for providing video streams or information derived therefrom the computing device 101 and the additional computing devices 151 over the network 125 to the SEE evaluations system 115).

In some embodiments, the SEE evaluation system 115 is used for an enterprise system. For example, an enterprise may subscribe to or otherwise utilize the SEE evaluation system 115 for allowing users of the enterprise to monitor their eye ergonomics and improve eye health. As used herein, the term “enterprise system” is intended to be construed broadly to include any group of systems or other computing devices. For example, the computing device 101, the additional computing devices 151 and IT assets of an IT infrastructure on which the SEE evaluation system 115 runs (e.g., in the information processing system 150 of FIG. 1B) may provide a portion of one or more enterprise systems. In some embodiments, an enterprise system includes one or more data centers, cloud infrastructure comprising one or more clouds, etc. A given enterprise system, such as cloud infrastructure, may host assets that are associated with multiple enterprises (e.g., two or more different businesses, organizations or other entities).

The computing device 101 and the additional computing devices 151 may be devices which are utilized by members of one or more enterprises, in any combination. Such devices are examples of what are more generally referred to herein as “processing devices.” Some of these processing devices are also generally referred to herein as “computers.” The computing device 101 and the additional computing devices 151 in some embodiments comprise respective computers associated with a particular company, organization or other enterprise. Thus, the computing device 101 and the additional computing devices 151 may be considered examples of assets of an enterprise system. In addition, at least portions of the information processing systems 100 and 150 may also be referred to herein as collectively comprising one or more “enterprises.” Numerous other operating scenarios involving a wide variety of different types and arrangements of processing nodes are possible, as will be appreciated by those skilled in the art.

The network 125 is assumed to comprise a global computer network such as the Internet, although other types of networks can be part of the network 125, including a wide area network (WAN), a local area network (LAN), a satellite network, a telephone or cable network, a cellular network, a wireless network such as a WiFi or WiMAX network, or various portions or combinations of these and other types of networks.

Although not explicitly shown in FIGS. 1A and 1B, one or more input-output devices such as keyboards, displays or other types of input-output devices may be used to support one or more user interfaces to the computing device 101, the additional computing devices 151 and the SEE evaluation system 115, as well as to support communication between such components and other related systems and devices not explicitly shown.

In some embodiments, such as in the information processing system 150 of FIG. 1B, the SEE evaluation system 115 may be provided as a cloud or web-based service that is accessible by one or more of the computing device 101 and the additional computing devices 151 to allow users thereof to manage their eye ergonomics. The computing device 101 and the additional computing devices 151 may be configured to access or otherwise utilize the SEE evaluation system 115 (e.g., to obtain real-time evaluations of eye ergonomics, to obtain reports or statistics related to eye ergonomics over some defined period of time, which may be broken down based on the tasks that users are performing on the computing device 101 and the additional computing devices 151, etc.). In some embodiments, one or more of the computing device 101 and the additional computing devices 151 are assumed to be associated with managers or other authorized personnel responsible for managing a set of users of an enterprise, organization or other entity. In some embodiments, the computing devices 101 and the additional computing devices 151 comprise IT assets which are owned or operated by the same enterprise that operates the SEE evaluation system 115. In other embodiments, the computing device 101 and the additional computing devices 151 may be owned or operated by one or more enterprises different than the enterprise which operates the SEE evaluation system 115 (e.g., a first enterprise provides eye ergonomics evaluation functionality and support for multiple different customers, businesses, etc.). Various other examples are possible.

In some embodiments, the computing devices 101 and the additional computing devices 151 may implement host agents that are configured for automated transmission of information with the SEE evaluation system 115 regarding eye usage or eye ergonomics while users thereof are performing different tasks. For example, eye usage or eye ergonomics evaluation provided by the SEE evaluation system 115 may be an “opt-in” feature that users of the computing device 101 and the additional computing devices 151 may selectively enable when desired. In some cases, an owner or operator of the computing device 101 and/or the additional computing devices 151 may automatically enroll in active eye usage or eye ergonomics evaluation for one or more designated users of the computing device 101 and/or the additional computing devices 151, when the computing device 101 and/or the additional computing devices 151 are being used for certain tasks (e.g., when an employee of an enterprise uses a computing device to perform work tasks, the enterprise may automatically activate the eye usage or eye ergonomics evaluation by the SEE evaluation system 115, etc.). It should be noted that a “host agent” as this term is generally used herein may comprise an automated entity, such as a software entity running on a processing device. Accordingly, a host agent need not be a human entity.

The SEE evaluation system 115 in the embodiments of FIGS. 1A and 1B is assumed to be implemented using at least one processing device. Each such processing device generally comprises at least one processor and an associated memory, and implements one or more functional modules or logic for controlling certain features of the SEE evaluation system 115. In the embodiments of FIGS. 1A and 1B, the SEE evaluation system 115 implements intelligent eye tracking logic 117, eye ergonomics analysis logic 119, eye ergonomics recommendation logic 121 and a SEE database 123. The intelligent eye tracking logic 117 is configured to identify when a user is viewing the display screen 102 of the computing device 101 (or a display screen of one of the additional computing devices 151) such as through facial recognition techniques, and tracks the eyes of the user.

The eye ergonomics analysis logic 119 is configured to utilize the eye tracking data in order to evaluate eye usage behavior of the user. This may include determining various aspects of eye ergonomics, including but not limited to eye distance, viewing angle, screen time, blink rate, etc.

The eye ergonomics recommendation logic 121 is configured to utilize the evaluated eye usage behavior of the user to take various action, such as generating warnings, alerts or other notifications to the user (e.g., using one or more of the visual indicators 109, the audio indicators 111 and the additional indicators 113) characterizing overall eye ergonomics or specific eye ergonomics factors (e.g., as good/poor, good/acceptable/poor, etc.). In some embodiments, such notifications include recommendations for improving eye ergonomics (e.g., moving closer or farther from the display screen 102, changing a viewing angle to the display screen 102, taking a screen time break, adjusting a blink rate, etc.). The eye ergonomics recommendation logic 121 may, in some cases, be configured to adjust a rate of delivery or a type of the notifications based on the severity of any detected eye ergonomics issues. This may include increasing a frequency of changing a type of indicator (e.g., from visual indicators 109 to audio indicators 111 or vice versa, increasing the size or location of notifications output on the display screen 102, etc.) used for delivery of warnings, alerts or other notifications the longer the user ignores eye ergonomics recommendations. Further, in some embodiments the eye ergonomics recommendation logic 121 may generate a report or summary of eye ergonomics evaluations for one or multiple users, possibly broken down according to various characteristics such as how eye ergonomics changes for one or more users depending on the tasks that such users are performing.

The SEE database 123 may be configured to store and record various information that is utilized or generated by the SEE evaluation system 115. Such information may include, for example, eye usage or eye ergonomics evaluations or reports. The SEE database 123 may be implemented utilizing one or more storage systems. The term “storage system” as used herein is intended to be broadly construed. A given storage system, as the term is broadly used herein, can comprise, for example, content addressable storage, flash-based storage, network-attached storage (NAS), storage area networks (SANs), direct-attached storage (DAS) and distributed DAS, as well as combinations of these and other storage types, including software-defined storage. Other particular types of storage products that can be used in implementing storage systems in illustrative embodiments include all-flash and hybrid flash storage arrays, software-defined storage products, cloud storage products, object-based storage products, and scale-out NAS clusters. Combinations of multiple ones of these and other storage products can also be used in implementing a given storage system in an illustrative embodiment.

At least portions of the intelligent eye tracking logic 117, the eye ergonomics analysis logic 119 and the eye ergonomics recommendation logic 121 may be implemented at least in part in the form of software that is stored in memory and executed by a processor.

It is to be appreciated that the particular arrangements of the computing device 101, the additional computing devices 151 and the SEE evaluation system 115 illustrated in the FIG. 1A and FIG. 1B embodiments are presented by way of example only, and alternative arrangements can be used in other embodiments. For example, the SEE evaluation system 115 may in some embodiments be implemented at least in part internal to and at least in part external to one or more of the computing device 101 and the additional computing devices 151.

The SEE evaluation system 115 and other portions of the information processing system 100, as will be described in further detail below, may be part of cloud infrastructure.

The SEE evaluation system 115 and other components of the information processing system 100 in the FIG. 1 embodiment are assumed to be implemented using at least one processing platform comprising one or more processing devices each having a processor coupled to a memory. Such processing devices can illustratively include particular arrangements of compute, storage and network resources.

The computing devices 101, the additional computing devices 151 and the SEE evaluation system 115 or components thereof (e.g., the intelligent eye tracking logic 117, the eye ergonomics analysis logic 119, the eye ergonomics recommendation logic 121 and the SEE database 123) may be implemented on respective distinct processing platforms, although numerous other arrangements are possible. For example, in some embodiments such as in the information processing system 100 the SEE evaluation system 115 and the computing device 101 are implemented on the same processing platform.

The term “processing platform” as used herein is intended to be broadly construed so as to encompass, by way of illustration and without limitation, multiple sets of processing devices and associated storage systems that are configured to communicate over one or more networks. For example, distributed implementations of the information processing systems 100 and 150 are possible, in which certain components of the system reside in one data center in a first geographic location while other components of the system reside in one or more other data centers in one or more other geographic locations that are potentially remote from the first geographic location. Thus, it is possible in some implementations of the information processing systems 100 and 150 for the computing device 101, the additional computing devices 151 and the SEE evaluation system 115, or portions or components thereof, to reside in different data centers. Numerous other distributed implementations are possible. The SEE evaluation system 115 can also be implemented in a distributed manner across multiple data centers.

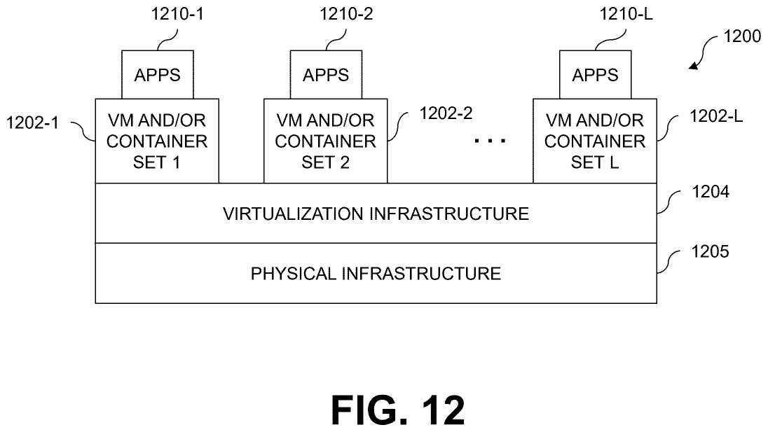

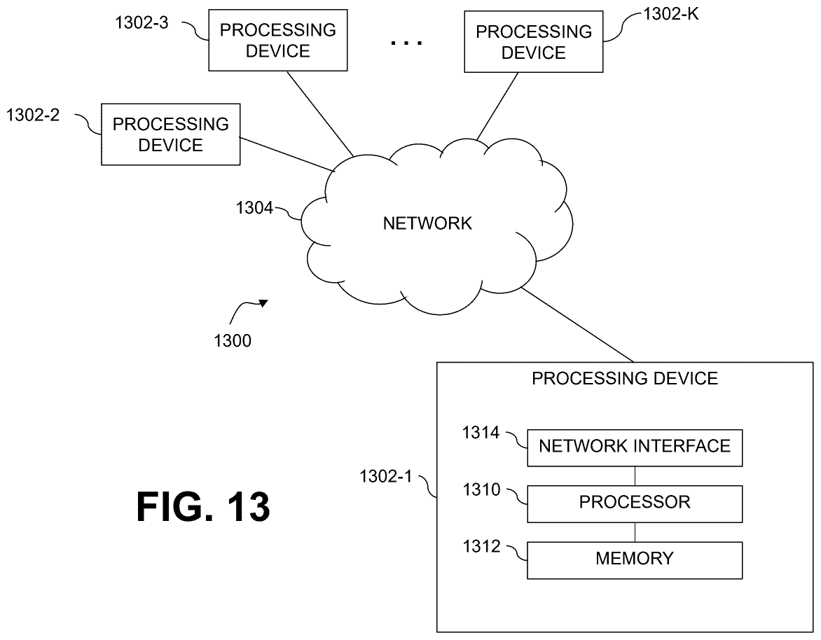

Additional examples of processing platforms utilized to implement the SEE evaluation system 115 and other components of the information processing systems 100 and 150 in illustrative embodiments will be described in more detail below in conjunction with FIGS. 12 and 13.

It is to be understood that the particular set of elements shown in FIGS. 1A and 1B for determining eye usage of a user and for evaluating conformance of the user with a set of eye usage patterns for viewing display screens are presented by way of illustrative example only, and in other embodiments additional or alternative elements may be used. Thus, another embodiment may include additional or alternative systems, devices and other network entities, as well as different arrangements of modules and other components.

It is to be appreciated that these and other features of illustrative embodiments are presented by way of example only, and should not be construed as limiting in any way.

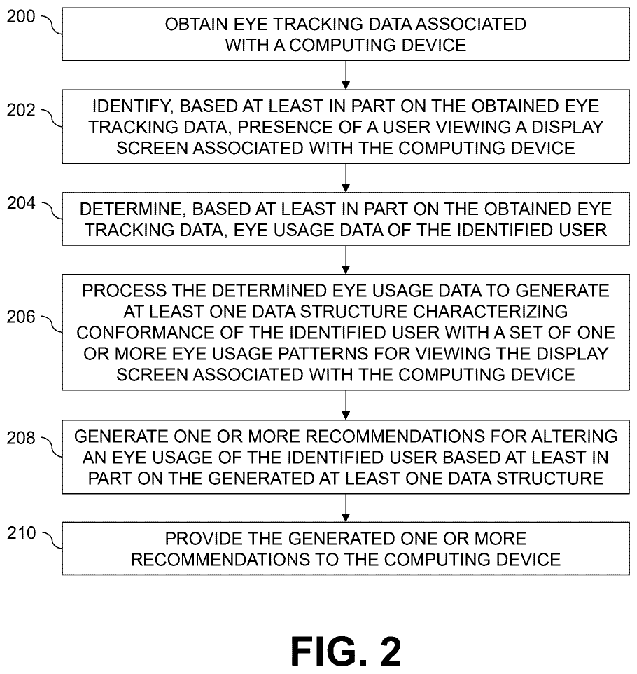

An exemplary process for determining eye usage of a user and for evaluating conformance of the user with a set of eye usage patterns for viewing display screens will now be described in more detail with reference to the flow diagram of FIG. 2. It is to be understood that this particular process is only an example, and that additional or alternative processes for determining eye usage of a user and for evaluating conformance of the user with a set of eye usage patterns for viewing display screens may be used in other embodiments.

In this embodiment, the process includes steps 200 through 210. These steps are assumed to be performed by the SEE evaluation system 115 utilizing the intelligent eye tracking logic 117, the eye ergonomics analysis logic 119 and the eye ergonomics recommendation logic 121. The process begins with step 200, obtaining eye tracking data associated with a computing device (e.g., the computing device 101, one of the additional computing devices 151). The eye tracking data associated with the computing device may be obtained from a video stream captured utilizing one or more imaging sensors (e.g., image sensors 103) associated with the computing device. The eye tracking data associated with the computing device may comprise data obtained from one or more depth sensors (e.g., depth sensors 105) associated with the computing device.

In step 202, presence of a user viewing a display screen (e.g., display screen 102) associated with the computing device is identified based at least in part on the obtained eye tracking data. Step 202 may include performing facial recognition of one or more faces detected in a video stream capture utilizing one or more imaging sensors associated with the computing device.

Eye usage data of the identified user is determined in step 204 based at least in part on the obtained eye tracking data. The determined eye usage data is processed in step 206 to generate at least one data structure characterizing conformance of the identified user with a set of one or more eye usage patterns for viewing the display screen associated with the computing device. One or more recommendations for altering an eye usage of the identified user are generated in step 208 based at least in part on the generated at least one data structure. The generated one or more recommendations are provided to the computing device in step 210. Step 210 may include activating one or more indicators associated with the computing device, the one or more indicators comprising at least one of a visual indicator (e.g., visual indicators 109) separate from the display screen associated with the computing device, a designated portion of the display screen associated with the computing device, and one or more audio indicators (e.g., audio indicators 111) associated with the computing device.

In some embodiments, step 204 includes determining a screen distance between one or more eyes of the identified user and the display screen associated with the computing device, the set of one or more eye usage patterns for viewing the display screen associated with the computing device comprises a recommended screen distance range, and wherein responsive to the generated at least one data structure indicating that the determined screen distance is outside the recommended screen distance range, at least one of the recommendations generated in step 208 comprises instructions for altering the determined screen distance to be within the recommended screen distance range.

In some embodiments, step 204 also or alternatively includes determining a viewing angle between one or more eyes of the identified user and the display screen associated with the computing device, the set of one or more eye usage patterns for viewing the display screen associated with the computing device comprises a recommended viewing angle range, and wherein responsive to the generated at least one data structure indicating that the determined viewing angle is outside the recommended viewing angle range, at least one of the recommendations generated in step 208 comprises instructions for altering the determined viewing angle to be within the recommended viewing angle range.

In some embodiments, step 204 further or alternatively includes determining a duration of time that one or more eyes of the identified user have been viewing the display screen associated with the computing device without an interruption lasting at least a threshold duration of time, the set of one or more eye usage patterns for viewing the display screen associated with the computing device comprises a recommended uninterrupted screen time duration, and wherein responsive to the generated at least one data structure indicating that the determined duration is less than the recommended uninterrupted screen time duration, at least one of the recommendations generated in step 208 comprises instructions for taking a screen time break lasting at least the threshold duration of time.

In some embodiments, step 204 further or alternatively includes determining a blinking rate of one or more eyes of the identified user, the set of one or more eye usage patterns for viewing the display screen associated with the computing device comprises a recommended blinking rate range, and wherein responsive to the generated at least one data structure indicating that the determined blinking rate is outside the recommended blinking rate range, at least one of the recommendations generated in step 208 comprises instructions for altering the determined blinking rate to be within the recommended blinking rate range.

The particular processing operations and other system functionality described in conjunction with the flow diagram of FIG. 2 are presented by way of illustrative example only, and should not be construed as limiting the scope of the disclosure in any way. Alternative embodiments can use other types of processing operations. For example, as indicated above, the ordering of the process steps may be varied in other embodiments, or certain steps may be performed at least in part concurrently with one another rather than serially. Also, one or more of the process steps may be repeated periodically, or multiple instances of the process can be performed in parallel with one another in order to implement a plurality of different processes for different computing devices or users viewing display screens thereof.

Functionality such as that described in conjunction with the flow diagram of FIG. 2 can be implemented at least in part in the form of one or more software programs stored in memory and executed by a processor of a processing device such as a computer or server. As will be described below, a memory or other storage device having executable program code of one or more software programs embodied therein is an example of what is more generally referred to herein as a “processor-readable storage medium.”

This millennium has seen an exponential increase in the use of digital tools, such as personal computers like laptops, desktops, etc. and other devices such as smartphones, tablets, smartwatches, video gaming systems, etc. These digital tools have proliferated into all walks of life, personal and professional. Further, use of such digital tools is prevalent across all age groups. Children may utilize digital tools for multiple hours for schoolwork, playing games, etc. Adults may spend most of the day at an office using various digital tools for work needs.

While digital tools have brought in various good changes for humankind, they can also present health-related hazards for people due to long hours of working with such digital tools. Such health-related hazards may be a result of, for example, bad sitting posture, bad viewing angles, bad lighting conditions, etc., which may cause irreparable damage to the human body if not corrected. The impact to the eyes of users is gradual, and the effect is long term. With exposure starting from an early age, the impact can be devastating. Prolonged and improper screen viewing habits can contribute to a range of eye-related issues, collectively known as Computer Vision Syndrome (CVS) or Digital Eye Strain. These and other issues are commonly associated with excessive screen time and poor viewing habits, such as sitting too close to a screen. Some common symptoms of CVS include eye strain and fatigue, dry eyes, excessive tearing, blurred or double vision, headaches, neck, shoulder or back pain, difficulty focusing, sensitivity to light, etc. There are some options for mitigating these issues, such as the use of antiglare glasses that help cut down the radiation to the eyes. Such options, however, are not sufficient to fully address these issues.

Illustrative embodiments provide technical solutions for a smart eye ergonomics (SEE) evaluation framework that can be run on various computing devices (e.g., laptops, desktops, tables, smartphone, smartwatches, video gaming systems, etc.) to help ensure that users thereof are exercising proper eye ergonomics. Proper eye ergonomics may include, for example, viewing a display screen at a proper distance, with a proper viewing angle, for a proper duration of time, with a proper blinking rate, etc. The SEE evaluation framework can analyze the eye ergonomics of a user, and generate recommendations for improving the user's eye ergonomics (e.g., such as with visual indicators showing whether a user is currently exercising good or poor eye ergonomics, notifications of how to improve eye ergonomics, generating reports or summaries of eye ergonomics of the user which may be tailored to different devices that a given user utilizes, different tasks that the given user performs, etc.). In some embodiments, the SEE evaluation framework leverages existing or available hardware of computing devices (e.g., cameras or other sensors) and utilizes computer vision techniques for evaluating eye ergonomics of users.

In some embodiments, the SEE evaluation framework provides various “checker” tools for different aspects or components of eye ergonomics, such as screen distance, viewing angle, screen time, and eye blink rate. The screen distance checker tool is configured to calculate the eye distance of the eyes of the user to a display screen of a computing device (e.g., in order to determine whether the user has a proper positioning based on the focal length for protection). The viewing angle checker tool is configured to calculate the viewing angle of the eyes of the user to the display screen of the computing device (e.g., in order to determine whether the user has a proper viewing angle). The screen time checker tool is configured to determine an amount of time that the user has been viewing a display screen (e.g., in order to determine if the user should take a break). The eye blink rate checker tool is configured to determine a blink rate of the eyes of the user (e.g., in order to determine if the user is blinking at an appropriate rate). These various checker tools may leverage one or more sensors, which may be existing sensors that are part of the computing device or may be external sensors which are connected or configured for use with the computing device to evaluate eye ergonomics. The sensors utilized may include, but are not limited to, camera, infrared or other imaging sensors, depth sensors, etc. Many computing devices are equipped with one or more of such sensors (e.g., webcams for laptop or desktop computing devices, front-facing cameras or other sensors of smartphones, tablets, etc.).

The SEE evaluation framework may utilize various types of indicators to provide indications of the determined eye ergonomics to the user. In some embodiments, a single indicator is used to show an overall indication of eye ergonomics to the user. In other embodiments, multiple indicators are used to show different aspects of eye ergonomics to the user (e.g., screen distance, viewing angle, screen time, eye blink rate). In either case, the indicators may include visual, audio or other indicators. Visual indicators may include, for example, LEDs or portions of the display screen (e.g., including pop-up or other notifications, status bars, etc.). Audio indicators may include, for example, chimes or other tones which are output via speakers connected to the computing device. Other indicators may include reports or summaries which are delivered to the user of a computing device, or possibly to additional users (e.g., human resources or other staff of an enterprise, organization or other entity which are responsible for managing employees or other users of the enterprise, organization or other entity, teachers, parents or other staff which are responsible for students, children, etc.). Such reports or summaries may provide detailed evaluations, such as characterizing the eye ergonomics of a given user while the given user performs different tasks (e.g., the given user may exercise proper eye ergonomics while performing work tasks such as utilizing a word processing software, but exercises poor eye ergonomics while performing leisure tasks such as web browsing, gaming or watching videos, etc.).

In some embodiments, indicators are color-based, where “red” indicates a problem, “orange” indicates a warning, and “green” indicates no problem. Various thresholds may be used for determining whether to display a red, orange or green indicator for overall eye ergonomics or different aspects of eye ergonomics. For example, an overall eye ergonomics indicator may be based on a number of eye ergonomics aspects which indicate a problem, warning or no problem, whether any particular or threshold number of eye ergonomics aspects is severe or not, etc.

Eyes are an important part of the human body, and thus there is a need or duty for users to diligently care for eye health. With the advent of digital tools, as described above, there is an inordinate amount of abuse that the eyes are subjected to that can have long-lasting adverse impact on overall eye health. The increased use of digital tools has been associated with a rise in the prevalence of Digital Eye Strain also known as CVS. Many users report experiencing symptoms of Digital Eye Strain or CVS. Further, there is a significant association between increased screen time and myopia (nearsightedness) development as well as an increased risk of dry eye disease. In addition, users may blink significantly less often during computer tasks as compared to non-computer tasks. It is estimated that 50-90% of the people using personal computers are prone to have at least some symptoms of Digital Eye Strain or CVS. Further, it is estimated that 80% of children ages 10-17 experience symptoms such as blurry vision.

There have been some advancements in digital technologies which seek to mitigate the risks of Digital Eye Strain or CVS, such as blue light filters and other improvements. Such advancements, however, are still unable to address all the issues. While it may not be feasible to stop users from using digital tools, there are various behavioral changes that could be adopted by users to mitigate these issues, including: maintaining an appropriate viewing distance from the display screen (e.g., the American Optometric Association suggests about 20-28 inches (in) or 50-70 centimeters (cm) from the eyes to the display screen); ensuring proper posture and ergonomic setup (e.g., positioning the display screen at a comfortable angle, slightly below eye level in order to have a viewing angle between) 15-45°; taking regular screen time breaks (e.g., the 20-20-20 rule, meaning for every 20 minutes take a 20 second break and look at an object at least 20 feet away); maintaining a proper blink rate (e.g., blinking frequently at least 12-15 times per minute, as staring at display screens for extended periods can lead to reduced blinking causing dryness and discomfort, where remembering to consciously blink more often can keep the eyes lubricated); and adjusting display screen settings (e.g., optimizing screen brightness, contrast and font size to reduce strain on the eyes).

While such behavioral modifications provide good guidelines which are simple to follow, it is difficult to ensure that users have the discipline to follow such guidelines while they are deeply immersed in whatever tasks they are using digital tools for. The technical solutions described herein provide an intelligent SEE evaluation framework which may be implemented in computing devices to provide timely assistance to users to ensure that they are following such guidelines or otherwise exercising proper or desired eye ergonomics (e.g., which may be defined or configured by a user, by a managing entity for a group of users, etc.). The SEE evaluation framework is also able to take remedial action if users are not exercising proper eye ergonomics, such as via prompts, reports, alerts, etc. provided to a given user (or to another user that manages or is otherwise responsible for the given user) to initiate corrective action. The SEE evaluation framework may continuously track the user's eyes using computer vision techniques, and derive the user's usage pattern to provide intervention when the user is not following some configured best practices for eye ergonomics. As such, use of the SEE evaluation framework can mitigate or prevent various conditions or issues such as Digital Eye Strain or CVS.

As discussed above, there is a need for intelligently tracking the movement of the eyes of users of digital tools, for identifying eye usage patterns (also referred to as eye ergonomics), and for alerting users when the identified eye usage patterns are determined to be sub-optimal. Even though users may be disciplined and aware of issues such as Digital Eye Strain or CVS, it is not always possible for a user to accurately and exactly track if they have deviated from best practices. Deteriorating health of the eyes and body in general may be attributed to the significant increase in screen time of users, and users not following best practices for eye ergonomics to mitigate such risks. In conventional approaches, users are not made aware of whether they are following eye ergonomics best practices for eye health. The technical solutions described herein provide the SEE evaluation framework which addresses these and other needs.

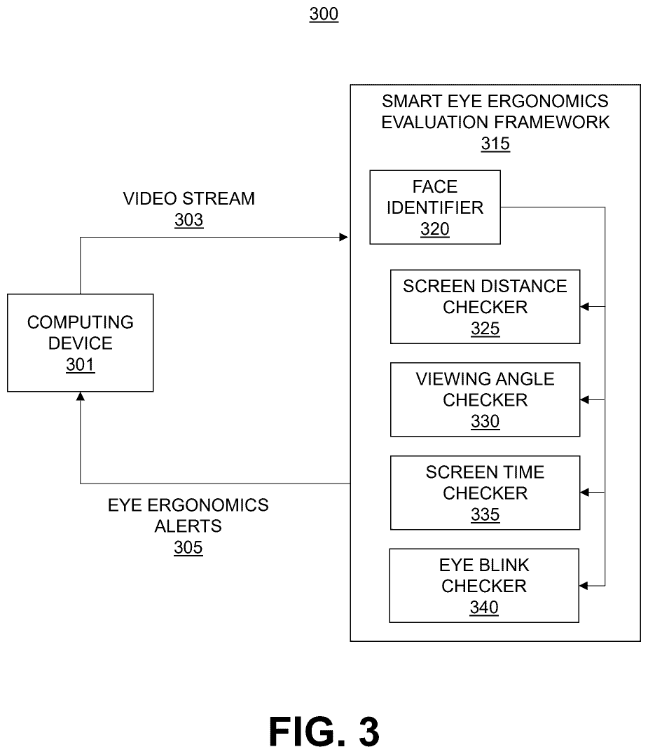

FIG. 3 shows a system 300 including a computing device 301 configured to utilize a SEE evaluation framework 315. In the description below, it may be assumed that the computing device 301 comprises a laptop computing device having an integrated webcam which is configured to provide a video stream 303 to the SEE evaluation framework 315. It should be appreciated, however, that the computing device 301 may be various other types of devices, such as a desktop computer with a monitor having an integrated webcam or external webcam attached thereto, a tablet or smartphone having a front-facing camera or other image sensor, etc. The SEE evaluation framework 315, in some embodiments, is implemented utilizing a lightweight TinyML or other machine learning model configured to run on smaller, less powerful devices (e.g., on the computing device 301, which may be a personal computing device such as a laptop computer as discussed above). It should be noted, however, that the SEE evaluation framework 315 in other embodiments may be implemented external to the computing device 301, such as on a server, a cloud computing platform, etc., which is accessible to the computing device 301 over one or more networks. In such cases, it may be desired for the computing device 301 to compress the video stream 303 prior to sending it to the SEE evaluation framework 315, or for the computing device 301 to perform some preprocessing of the video stream 303 (e.g., feature extraction, calculation of metrics such as screen distance, viewing angle, screen time, blink rate, etc.) in order to reduce the amount of data provided over the networks interconnecting the computing device 301 and the SEE evaluation framework 315.

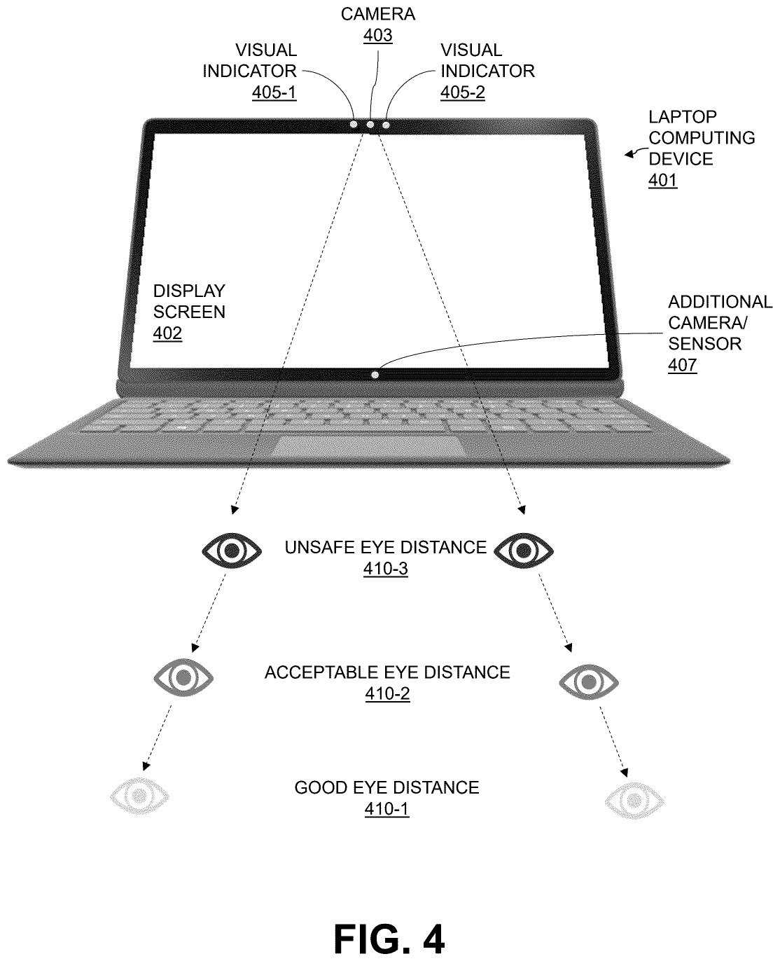

FIG. 4 shows a particular implementation of the computing device 301 as a laptop computing device 401, which has a camera 403 proximate a middle of a display screen 402 of the laptop computing device 401 above the top of the display screen 402, and one or more additional cameras or other sensors 407 proximate the middle of the display screen 402 of the laptop computing device 401 below the bottom of the display screen 402. The laptop computing device 401 also includes a set of visual indicators 405-1 and 405-2 (collectively, visual indicators 405) which are separate from the display screen 402 of the laptop computing device 401. The visual indicators 405 may comprise, for example, LEDs configured to display indications of overall eye ergonomics or specific eye ergonomics factors. Indications of overall eye ergonomics or specific eye ergonomics factors may also or alternatively be presented on the display screen 402 itself. In the example of FIG. 4, it is assumed that at least one of the visual indicators 405 presents information related to eye distance of a user of the laptop computing device 401. Here, different shades of gray are used to indicate different range of eye distance, including a good eye distance 410-1 (e.g., 20.1-30 in), an acceptable eye distance 410-2 (e.g., 16-20 in), and an unsafe eye distance 410-3 (e.g., less than 16 in). It should be noted, however, that these ranges are presented by way of example only and that embodiments are not limited to use with these specific value ranges.

Further, more or fewer than three ranges may be utilized. The different ranges may also be user-configured, such that a user can customize the alerts or other indications which are presented based on that user's individual preferences regarding screen distance. Similarly, a user may provide custom configurations of acceptable ranges of values for other eye ergonomic factors such as viewing angle, screen time, blink rate, etc.

The SEE evaluation framework 315 is configured to analyze the video stream 303 from the computing device 301 (or information or metrics derived therefrom as discussed above) to determine whether a user of the computing device 301 is exercising best practices related to eye ergonomics, and for providing eye ergonomics alerts 305 or other intervention to the computing device 301 when needed. In some cases, for example, the SEE evaluation framework 315 may be configured to automatically control a display screen (e.g., adjusting display characteristics such as brightness, turning the display screen on and off, etc.) of the computing device 301 in order to enforce eye ergonomics best practices. In some embodiments, the SEE evaluation framework 315 can be configured so as to adjust a positioning of a display screen of the computing device 301 (e.g., such as using one or more actuators to adjust an angle of the display screen or a dock on which the computing device 301 rests, etc.). Many laptops and other types of computing devices are fitted with infrared emitters or infrared cameras or sensors, depth sensing cameras or sensors, etc., which may be used in place of or in addition to webcams to provide more accurate information on the distance (e.g., depth) of the subject or user from the display screen.

As shown in FIG. 3, the SEE evaluation framework 315 implements a face identifier 320 configured to identify a particular user based on face recognition technology. Once the user is recognized, various checker tools may be initiated to track and monitor various aspects of the eye ergonomics of the user, including a screen distance checker 325 (e.g., configured to evaluate the user's eye distance from the display screen of the computing device 301), a viewing angle checker 330 (e.g., configured to evaluate the user's viewing angle to the display screen of the computing device 301), a screen time checker 335 (e.g., configured to evaluate the user's duration of continuous viewing of the display screen of the computing device 301) and an eye blink checker 340 (e.g., configured to evaluate the user's eye blink rate, such as eye blinks per minute).

In some embodiments, the SEE evaluation framework 315 may utilize TensorFlow Lite as a base model for performing detection and prediction. The model may be pre-trained with publicly available datasets for faces (e.g., the Face Detection Data Set and Benchmark (FDDB) data set, the UMDFaces dataset, etc.) and eyes (e.g., the BioID Face database, the OpenEDS dataset, etc.) to cover various characteristics such as different sexes, age groups, geographies, ethnicities, with and without spectacles, etc. to enable the model to identify if the video stream includes a face (e.g., which means that a user is viewing the display screen of the computing device 301) and to later track the eyes of the user for further prediction.

The data may be prepared so that the functionalities of the SEE evaluation framework 315 work as expected. For example, multiple scenarios may be considered before actual prediction can happen. Such scenarios include, for example: that the user could have stepped away from the display screen, such that tracking and prediction should be paused for the duration that the user has stepped away from the display screen; that there may be multiple users viewing the screen, such that the face identifier 320 may determine the closest one among them to perform the next set of tracking; there could be some glare on the face due to light conditions which may be countered with appropriate enrichment techniques to ensure that the face and eyes can be tracked accurately; that a new user might take over from an earlier user, and the face identifier 320 may identify this change by comparing a frame of the video stream 303 with earlier frames of the video stream 303 and providing this information to the various checkers to act accordingly; etc.

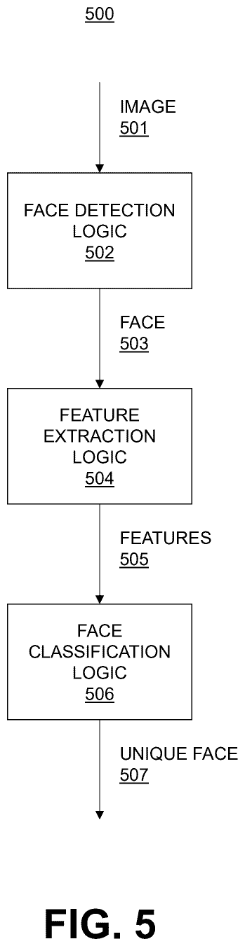

The face identifier 320 ensures that faces are identified in the video stream 303, and that face information is extracted from one or more frames of the video stream 303 with the right level of information for enabling accurate predictions to happen using the various checker tools. In some embodiments, the face identifier 320 utilizes FaceNet, a convolutional neural network (CNN)-based model for recognizing faces. The face identifier 320 is configured to extract key facial features from the image input (e.g., frames of the video stream 303), creates a vector call embedding, and uses a classifier (e.g., a SoftMax classifier) to identify different faces and changes in faces. FIG. 5 shows a process flow 500 for identifying and classifying faces which may be implemented by the face identifier 320. The process flow 500 includes providing an image 501 (e.g., a frame of the video stream 303 of FIG. 3) to face detection logic 502, which detects a face 503 in the image 501. The face 503 is then provided to feature extraction logic 504 configured to extract features 505 of the face 503. The features 505 are then used by face classification logic 506 to identify a unique face 507 (e.g., to classify the face 503 in the image 501 as belonging to a specific user).

The screen distance checker 325 is configured to evaluate a screen distance between eyes of the user and the display screen of the computing device 301. Appropriate viewing distance from the display screen is a critical factor for the health of the eyes. In some embodiments, a target viewing distance is configured in the SEE evaluation framework 315 (e.g., such as an optimal viewing distance of 20-30 in from the display screen). Anything closer or farther than this configured target viewing distance may be flagged and cause generation of one of the eye ergonomics alerts 305. For example, an eye ergonomics alert may indicate that the user is closer or farther than the configured target viewing distance, which may cause harm to the eyes of the user. The screen distance checker 325 will determine if the identified user is viewing the display screen of the computing device 301 from an appropriate viewing distance, and provide one or more of the eye ergonomics alerts 305 in response (e.g., including potentially indicating that the screen distance is good/optimal or bad/suboptimal, or more granular such as good/optimal, ok/acceptable or bad/suboptimal, and if ok/acceptable or bad/suboptimal may further indicate how the user can remedy or improve this factor such as moving closer or farther from the display screen of the computing device 301, etc.).

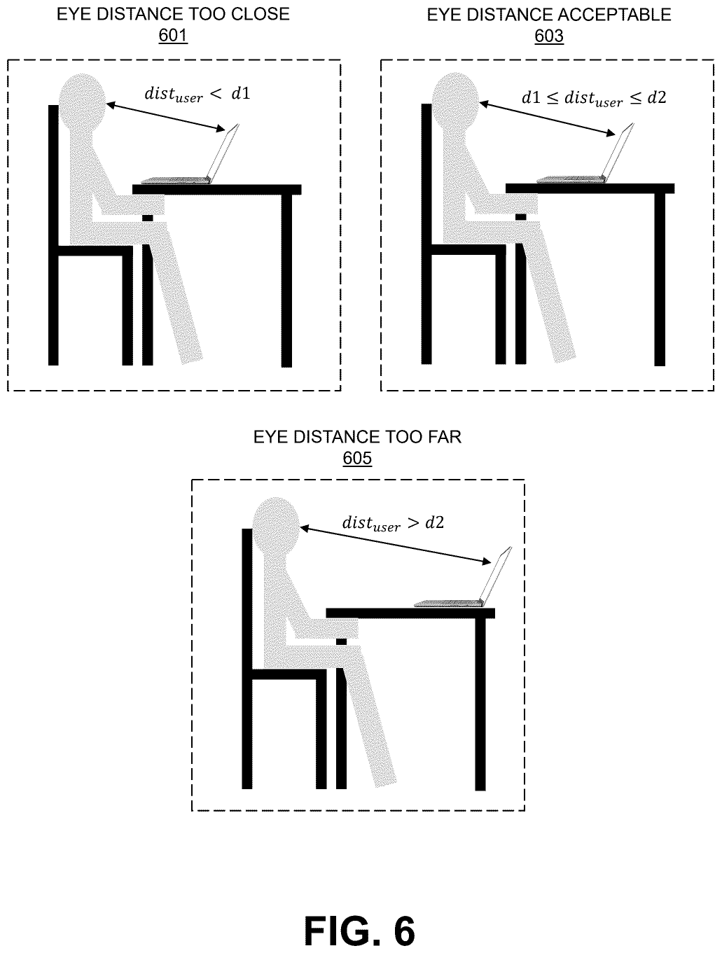

FIG. 6 shows visualizations of different screen distance ranges, including where the eye distance is too close 601 (e.g., where the eye distance of the user, denoted distᵤₛₑᵣ, is less than a first threshold distance d1), where the eye distance is acceptable 603 (e.g., where distᵤₛₑᵣ is greater than or equal to d1 but less than or equal to a second threshold distance d2), and where the eye distance is too far 605 (e.g., where distᵤₛₑᵣ is greater than d2).

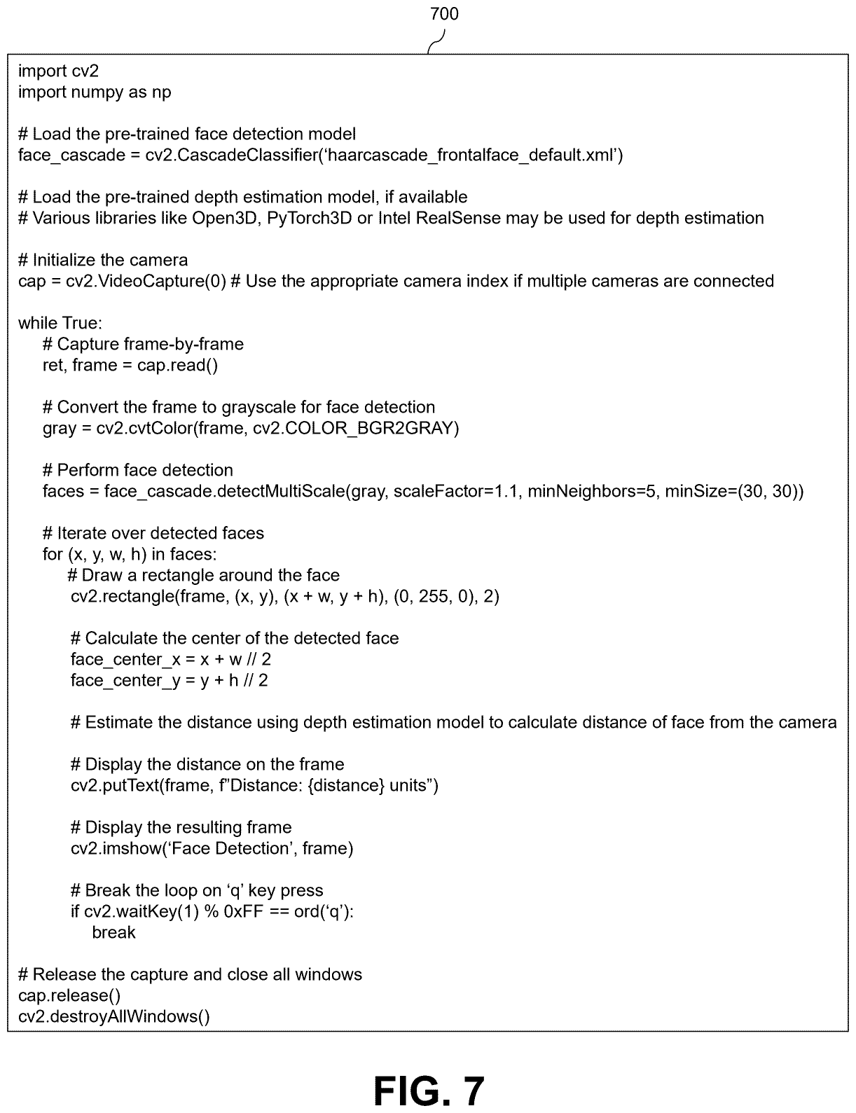

In some embodiments, the screen distance checker 325 uses depth estimation techniques based on monocular images captured from the video stream 303. The depth estimation techniques may use OpenCV, PyTorch3D or other open-source computer vision libraries to determine the distance of the user's face from the display screen of the computing device 301. FIG. 7 shows pseudocode 700 which may be used by the screen distance checker 325 for deriving the screen viewing distance.

The viewing angle checker 330 is configured to evaluate a viewing angle between the eyers of the user and the display screen of the computing device 301. An appropriate viewing angle from the display screen is a critical factor not just for the health of the eyes, but also for the neck and shoulders of the user. It is recommended that the top of the monitor or other display screen should be slightly lower than eye level, so that the user is looking down at an angle of about 15° with a viewing angle of 30° spread. Any lesser or greater angle may cause harm. The viewing angle checker 330 is configured to determine if the user has some configured target viewing angle, and provides one or more of the eye ergonomics alerts 305 in response to determining that the users has a viewing angle that is outside the configured target viewing angle.

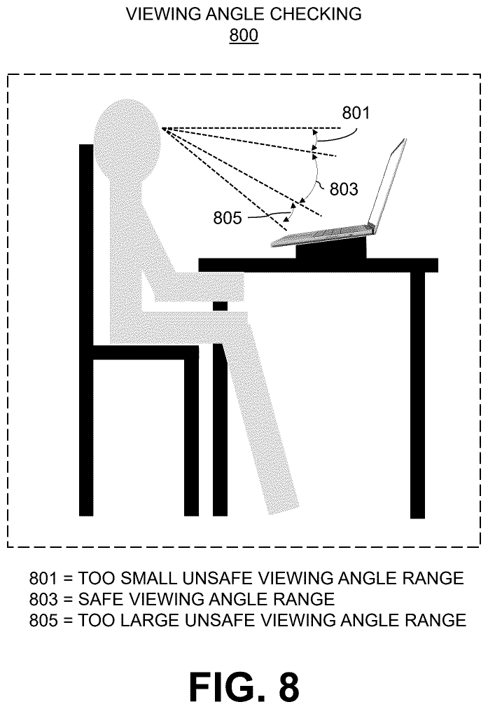

FIG. 8 shows a visualization 800 of different viewing angle ranges, including where the viewing angle is in a too small unsafe viewing angle range 801 (e.g., where the viewing angle of the user is less than a first viewing angle threshold, θ1, which may be 15°), where the viewing angle is in a safe viewing angle range 803 (e.g., where the viewing angle is equal to or greater than θ1 and less than or equal to a second viewing angle threshold θ2, which may be 45°), and where the viewing angle is in a too large unsafe viewing angle range 805 (e.g., where the viewing angle is greater than θ2).

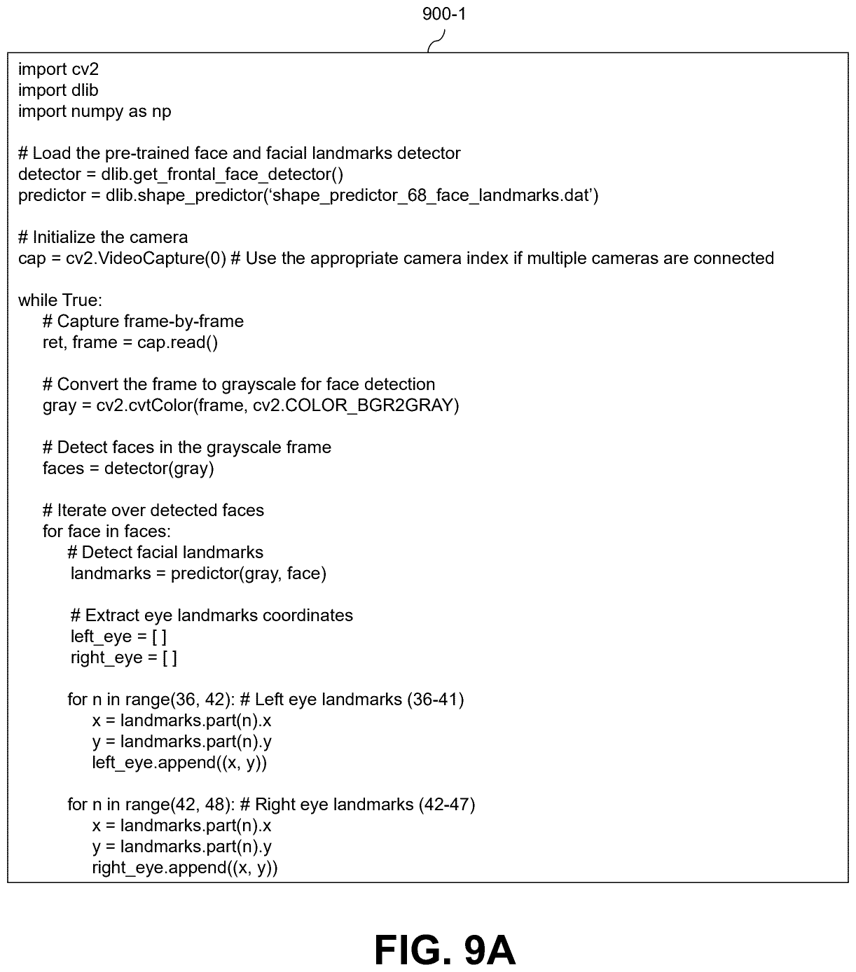

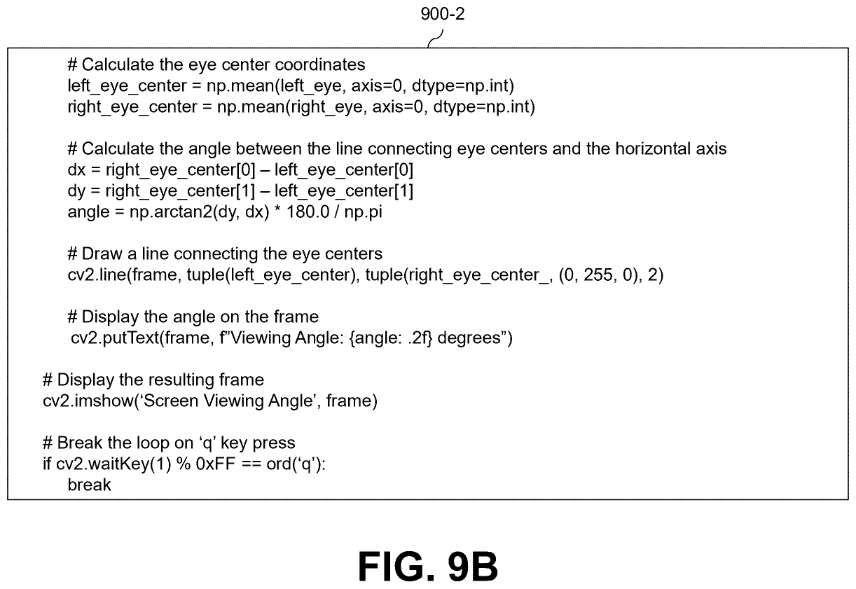

In some embodiments, the viewing angle checker 330 may use a combination of face detection, eye gaze estimation techniques and geometric calculations to determine viewing angle of the user. Such techniques may leverage the capabilities provided in various open source libraries, including but not limited to OpenCV, DLIB and PyTorch3D to determine the viewing angle of the user's eyes from the top of the display screen of the computing device 301. FIGS. 9A and 9B show pseudocode 900-1 and 900-2 (collectively, pseudocode 900) for deriving the screen viewing angle.

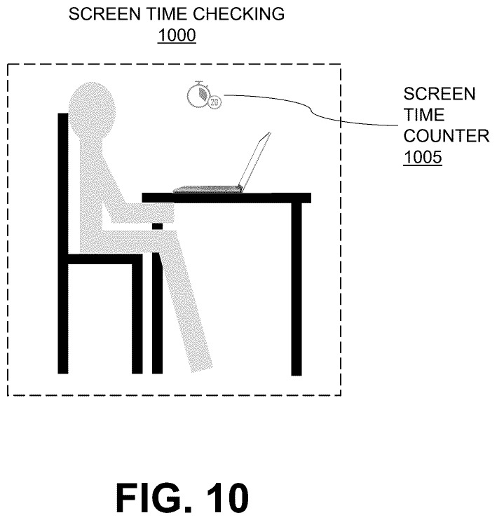

The screen time checker 335 is configured to evaluate a screen time or duration that the user is or has been viewing the display screen of the computing device 301. Taking frequent screen time breaks is another important aspect of eye ergonomics. The 20-20-20 rule, for example, specifies that every 20 minutes the user should take a 20 second break and look at an object at least 20 feet away. These and other screen time rules may be configured in the SEE evaluation framework 315 as desired. The screen time checker 335 will track a user's face for a specified duration of time (e.g., 20 minutes) and, if the user's face is determined to be available uninterrupted for that specified duration of time, then one or more of the eye ergonomics alerts 305 may be delivered to indicate that the user should take a screen time break. The alerts may have different levels, based on different screen time thresholds (e.g., a warning when a first screen time threshold is reached, an alert when a second screen time threshold is reached, turning off the display screen of the computing device 301 when a third screen time threshold is reached, etc.). If the user ignores the screen time eye ergonomics alerts 305 and continues to use the display screen of the computing device 301, then the eye ergonomics alerts 305 may continue to be delivered (e.g., every 10 minutes, at successively shorter intervals, etc.) until it is determined that the user steps away from the display screen of the computing device 301. Any time that the screen time checker 335 determines that the user has stepped away from the display screen of the computing device 301 for at least some threshold duration of time (e.g., 20 seconds), then the timer or counter of the continuous screen time may be reset. Further, whenever it is determined that the user viewing the display screen of the computing device 301 has changed (e.g., one user takes over from an earlier user), the timer or counter may similarly be reset. FIG. 10 shows a visualization 1000 of screen time checking, including a timer or counter 1005 of continuous screen time.

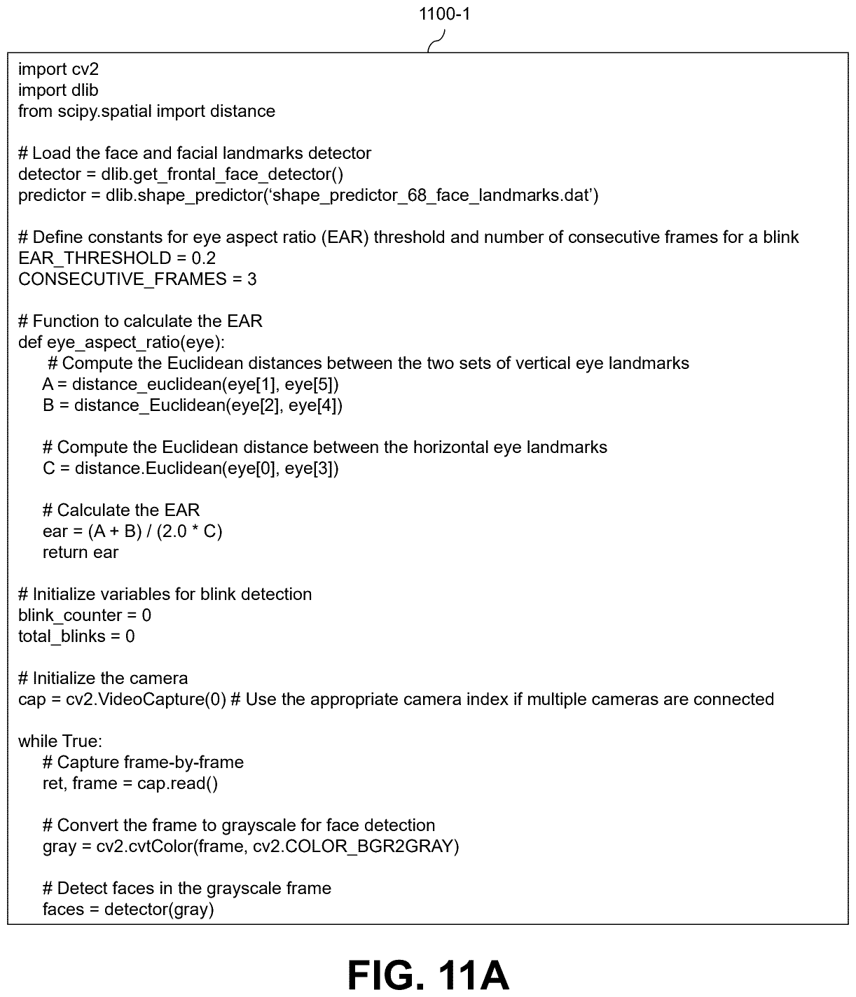

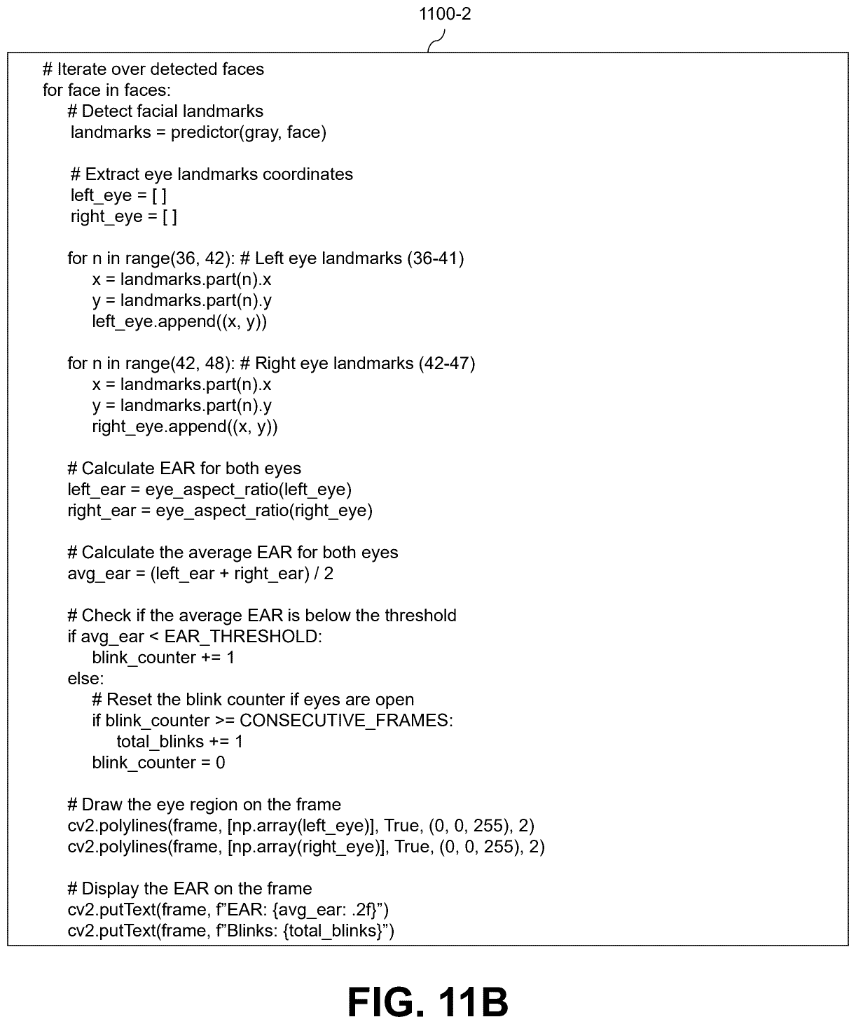

The eye blink checker 340 is configured to evaluate a blink rate of the user viewing the display screen of the computing device 301. Blinking of the eyes is an important function which helps to keep eyes hydrated. An average human eye, for example, should blink 12-15 times per minute. Staring at display screens for extended periods can lead to reduced blinking, causing dryness and discomfort. The eye blink checker 340 is configured to check the number of times that the eyes of the user blink over some time period (e.g., a minute), and the eye blink counter may be reset after each expiration of that time period (e.g., each minute). In some embodiments, the eye blink checker 340 determines an average blink rate of the user over a moving or sliding time window that is longer than each time period (e.g., a 5 minute sliding time window). If the user's blink rate is outside some configured target blink rate frequency range, then one or more of the eye ergonomics alerts 305 may be delivered recommending that the user, for example, increases their blink rate. In some embodiments, the eye blink checker 340 uses facial landmark detection techniques, such as those provided by open source libraries such as OpenCV and DLIB to identify eye blinks. FIGS. 11A and 11B show pseudocode 1100-1 and 1100-2 (collectively, pseudocode 1100) which may be implemented by the eye blink checker 340 for identifying eye blinks and blink rate.

It should be noted that each of the checker tools of the SEE evaluation framework 315 may be configured to provide timely notifications of eye ergonomics status, particularly warnings and alerts, so that users can take appropriate action to improve their eye ergonomics. The particular notification mechanism used may vary depending on the device type and indicators equipped on the computing device 301, as well as operating system capabilities of the computing device 301.

Illustrative embodiments provide technical solutions for tracking a user's eye ergonomics conditions while viewing display screens of computing devices. The technical solutions leverage computer vision techniques and hardware such as cameras and other sensors equipped on computing devices to provide real-time recommendations with respect to violation of configured eye ergonomics best practices (e.g., maintaining proper viewing distance, viewing angle, blink rate, taking frequent breaks, using the right display settings, etc.).

It should be noted that the data collected for evaluating eye ergonomics (e.g., facial data) may in some embodiments not be shared beyond the edge or realm of the computing device that the user is viewing. All processing may be handled locally (e.g., as in the system 100 of FIG. 1A) deployed on the edge, and data may not be stored or shared outside of it (e.g., in other devices, backend servers, etc.). Even the video stream does not have to be stored, as it is only needed to extract the facial data with the rest being discarded. Thus, the technical solutions may be compliant with various consumer privacy regulations related to continuous monitoring of the user's face and surrounding environment. In embodiments where processing is handled remotely (e.g., as in the system 150 of FIG. 1B), then suitable mechanisms such as encryption, anonymization, etc. may be adopted to address any potential privacy concerns. Further, the technical solutions may provide user opt-in functionality, allowing users to control whether the SEE evaluation framework is installed or used at any given time.

When processing is handled locally (e.g., as in the system 100 of FIG. 1A), there may be some impact to the performance of the computing device due to the continuous capture and analysis of a video stream. Some embodiments may minimize or reduce such performance impacts by leveraging lightweight machine learning models such as TinyML.

It is to be appreciated that the particular advantages described above and elsewhere herein are associated with particular illustrative embodiments and need not be present in other embodiments. Also, the particular types of information processing system features and functionality as illustrated in the drawings and described above are exemplary only, and numerous other arrangements may be used in other embodiments.

Illustrative embodiments of processing platforms utilized to implement functionality for determining eye usage of a user and for evaluating conformance of the user with a set of eye usage patterns for viewing display screens will now be described in greater detail with reference to FIGS. 12 and 13. Although described in the context of system 100, these platforms may also be used to implement at least portions of other information processing systems in other embodiments.

FIG. 12 shows an example processing platform comprising cloud infrastructure 1200. The cloud infrastructure 1200 comprises a combination of physical and virtual processing resources that may be utilized to implement at least a portion of the information processing system 100 in FIG. 1. The cloud infrastructure 1200 comprises multiple virtual machines (VMs) and/or container sets 1202-1, 1202-2, . . . 1202-L implemented using virtualization infrastructure 1204. The virtualization infrastructure 1204 runs on physical infrastructure 1205, and illustratively comprises one or more hypervisors and/or operating system level virtualization infrastructure. The operating system level virtualization infrastructure illustratively comprises kernel control groups of a Linux operating system or other type of operating system.

The cloud infrastructure 1200 further comprises sets of applications 1210-1, 1210-2, . . . 1210-L running on respective ones of the VMs/container sets 1202-1, 1202-2, . . . 1202-L under the control of the virtualization infrastructure 1204. The VMs/container sets 1202 may comprise respective VMs, respective sets of one or more containers, or respective sets of one or more containers running in VMs.

In some implementations of the FIG. 12 embodiment, the VMs/container sets 1202 comprise respective VMs implemented using virtualization infrastructure 1204 that comprises at least one hypervisor. A hypervisor platform may be used to implement a hypervisor within the virtualization infrastructure 1204, where the hypervisor platform has an associated virtual infrastructure management system. The underlying physical machines may comprise one or more distributed processing platforms that include one or more storage systems.

In other implementations of the FIG. 12 embodiment, the VMs/container sets 1202 comprise respective containers implemented using virtualization infrastructure 1204 that provides operating system level virtualization functionality, such as support for Docker containers running on bare metal hosts, or Docker containers running on VMs. The containers are illustratively implemented using respective kernel control groups of the operating system.

As is apparent from the above, one or more of the processing modules or other components of system 100 may each run on a computer, server, storage device or other processing platform element. A given such element may be viewed as an example of what is more generally referred to herein as a “processing device.” The cloud infrastructure 1200 shown in FIG. 12 may represent at least a portion of one processing platform. Another example of such a processing platform is processing platform 1300 shown in FIG. 13.

The processing platform 1300 in this embodiment comprises a portion of system 100 and includes a plurality of processing devices, denoted 1302-1, 1302-2, 1302-3, . . . 1302-K, which communicate with one another over a network 1304.

The network 1304 may comprise any type of network, including by way of example a global computer network such as the Internet, a WAN, a LAN, a satellite network, a telephone or cable network, a cellular network, a wireless network such as a WiFi or WiMAX network, or various portions or combinations of these and other types of networks.

The processing device 1302-1 in the processing platform 1300 comprises a processor 1310 coupled to a memory 1312.

The processor 1310 may comprise a microprocessor, a microcontroller, an application-specific integrated circuit (ASIC), a field-programmable gate array (FPGA), a central processing unit (CPU), a graphical processing unit (GPU), a tensor processing unit (TPU), a video processing unit (VPU) or other type of processing circuitry, as well as portions or combinations of such circuitry elements.

The memory 1312 may comprise random access memory (RAM), read-only memory (ROM), flash memory or other types of memory, in any combination. The memory 1312 and other memories disclosed herein should be viewed as illustrative examples of what are more generally referred to as “processor-readable storage media” storing executable program code of one or more software programs.

Articles of manufacture comprising such processor-readable storage media are considered illustrative embodiments. A given such article of manufacture may comprise, for example, a storage array, a storage disk or an integrated circuit containing RAM, ROM, flash memory or other electronic memory, or any of a wide variety of other types of computer program products. The term “article of manufacture” as used herein should be understood to exclude transitory, propagating signals. Numerous other types of computer program products comprising processor-readable storage media can be used.

Also included in the processing device 1302-1 is network interface circuitry 1314, which is used to interface the processing device with the network 1304 and other system components, and may comprise conventional transceivers.

The other processing devices 1302 of the processing platform 1300 are assumed to be configured in a manner similar to that shown for processing device 1302-1 in the figure.

Again, the particular processing platform 1300 shown in the figure is presented by way of example only, and system 100 may include additional or alternative processing platforms, as well as numerous distinct processing platforms in any combination, with each such platform comprising one or more computers, servers, storage devices or other processing devices.

For example, other processing platforms used to implement illustrative embodiments can comprise converged infrastructure.

It should therefore be understood that in other embodiments different arrangements of additional or alternative elements may be used. At least a subset of these elements may be collectively implemented on a common processing platform, or each such element may be implemented on a separate processing platform.

As indicated previously, components of an information processing system as disclosed herein can be implemented at least in part in the form of one or more software programs stored in memory and executed by a processor of a processing device. For example, at least portions of the functionality for determining eye usage of a user and for evaluating conformance of the user with a set of eye usage patterns for viewing display screens as disclosed herein are illustratively implemented in the form of software running on one or more processing devices.

It should again be emphasized that the above-described embodiments are presented for purposes of illustration only. Many variations and other alternative embodiments may be used. For example, the disclosed techniques are applicable to a wide variety of other types of information processing systems, processing devices, cameras, sensors, etc. Also, the particular configurations of system and device elements and associated processing operations illustratively shown in the drawings can be varied in other embodiments. Moreover, the various assumptions made above in the course of describing the illustrative embodiments should also be viewed as exemplary rather than as requirements or limitations of the disclosure. Numerous other alternative embodiments within the scope of the appended claims will be readily apparent to those skilled in the art.

What is claimed is:

1. An apparatus comprising:
at least one processing device comprising a processor coupled to a memory;
the at least one processing device being configured:
to obtain eye tracking data associated with a computing device;
to identify, based at least in part on the obtained eye tracking data, presence of a user viewing a display screen associated with the computing device;
to determine, based at least in part on the obtained eye tracking data, eye usage data of the identified user;
to process the determined eye usage data to generate at least one data structure characterizing conformance of the identified user with a set of one or more eye usage patterns for viewing the display screen associated with the computing device;
to generate one or more recommendations for altering an eye usage of the identified user based at least in part on the generated at least one data structure; and
to provide the generated one or more recommendations to the computing device.

2. The apparatus of claim 1 wherein the eye tracking data associated with the computing device is obtained from a video stream captured utilizing one or more imaging sensors associated with the computing device.

3. The apparatus of claim 1 wherein the eye tracking data associated with the computing device comprises data obtained from one or more depth sensors associated with the computing device.

4. The apparatus of claim 1 wherein identifying the presence of the user viewing the display screen associated with the computing device comprises performing facial recognition of one or more faces detected in a video stream capture utilizing one or more imaging sensors associated with the computing device.

5. The apparatus of claim 1 wherein determining the eye usage data of the identified user comprises determining a screen distance between one or more eyes of the identified user and the display screen associated with the computing device.

6. The apparatus of claim 5 wherein the set of one or more eye usage patterns for viewing the display screen associated with the computing device comprises a recommended screen distance range, and wherein responsive to the generated at least one data structure indicating that the determined screen distance is outside the recommended screen distance range, at least one of the generated one or more recommendations comprises instructions for altering the determined screen distance to be within the recommended screen distance range.

7. The apparatus of claim 1 wherein determining the eye usage data of the identified user comprises determining a viewing angle between one or more eyes of the identified user and the display screen associated with the computing device.

8. The apparatus of claim 7 wherein the set of one or more eye usage patterns for viewing the display screen associated with the computing device comprises a recommended viewing angle range, and wherein responsive to the generated at least one data structure indicating that the determined viewing angle is outside the recommended viewing angle range, at least one of the generated one or more recommendations comprises instructions for altering the determined viewing angle to be within the recommended viewing angle range.

9. The apparatus of claim 1 wherein determining the eye usage data of the identified user comprises determining a duration of time that one or more eyes of the identified user have been viewing the display screen associated with the computing device without an interruption lasting at least a threshold duration of time.

10. The apparatus of claim 9 wherein the set of one or more eye usage patterns for viewing the display screen associated with the computing device comprises a recommended uninterrupted screen time duration, and wherein responsive to the generated at least one data structure indicating that the determined duration is less than the recommended uninterrupted screen time duration, at least one of the generated one or more recommendations comprises instructions for taking a screen time break lasting at least the threshold duration of time.

11. The apparatus of claim 1 wherein determining the eye usage data of the identified user comprises determining a blinking rate of one or more eyes of the identified user.

12. The apparatus of claim 11 wherein the set of one or more eye usage patterns for viewing the display screen associated with the computing device comprises a recommended blinking rate range, and wherein responsive to the generated at least one data structure indicating that the determined blinking rate is outside the recommended blinking rate range, at least one of the generated one or more recommendations comprises instructions for altering the determined blinking rate to be within the recommended blinking rate range.

13. The apparatus of claim 1 wherein the at least one processing device is part of the computing device.

14. The apparatus of claim 1 wherein providing the generated one or more recommendations to the computing device comprises activating one or more indicators associated with the computing device, the one or more indicators comprising at least one of a visual indicator separate from the display screen associated with the computing device, a designated portion of the display screen associated with the computing device, and one or more audio indicators associated with the computing device.

15. A computer program product comprising a non-transitory processor-readable storage medium having stored therein program code of one or more software programs, wherein the program code when executed by at least one processing device causes the at least one processing device:
to obtain eye tracking data associated with a computing device;
to identify, based at least in part on the obtained eye tracking data, presence of a user viewing a display screen associated with the computing device;
to determine, based at least in part on the obtained eye tracking data, eye usage data of the identified user;
to process the determined eye usage data to generate at least one data structure characterizing conformance of the identified user with a set of one or more eye usage patterns for viewing the display screen associated with the computing device;
to generate one or more recommendations for altering an eye usage of the identified user based at least in part on the generated at least one data structure; and
to provide the generated one or more recommendations to the computing device.

16. The computer program product of claim 15 wherein identifying the presence of the user viewing the display screen associated with the computing device comprises performing facial recognition of one or more faces detected in a video stream capture utilizing one or more imaging sensors associated with the computing device.

17. The computer program product of claim 15 wherein determining the eye usage data of the identified user comprises:
determining a screen distance between one or more eyes of the identified user and the display screen associated with the computing device;
determining a viewing angle between one or more eyes of the identified user and the display screen associated with the computing device;
determining a duration of time that one or more eyes of the identified user have been viewing the display screen associated with the computing device without an interruption lasting at least a threshold duration of time; and
determining a blinking rate of one or more eyes of the identified user.

18. A method comprising:
obtaining eye tracking data associated with a computing device;
identifying, based at least in part on the obtained eye tracking data, presence of a user viewing a display screen associated with the computing device;
determining, based at least in part on the obtained eye tracking data, eye usage data of the identified user;
processing the determined eye usage data to generate at least one data structure characterizing conformance of the identified user with a set of one or more eye usage patterns for viewing the display screen associated with the computing device;
generating one or more recommendations for altering an eye usage of the identified user based at least in part on the generated at least one data structure; and
providing the generated one or more recommendations to the computing device;
wherein the method is performed by at least one processing device comprising a processor coupled to a memory.

19. The method of claim 18 wherein identifying the presence of the user viewing the display screen associated with the computing device comprises performing facial recognition of one or more faces detected in a video stream capture utilizing one or more imaging sensors associated with the computing device.

20. The method of claim 18 wherein determining the eye usage data of the identified user comprises:
determining a screen distance between one or more eyes of the identified user and the display screen associated with the computing device;
determining a viewing angle between one or more eyes of the identified user and the display screen associated with the computing device;
determining a duration of time that one or more eyes of the identified user have been viewing the display screen associated with the computing device without an interruption lasting at least a threshold duration of time; and
determining a blinking rate of one or more eyes of the identified user.
